(19) United States
(12) Plant Patent Application Publication
(10) Pub. No.: US 2017/0006755 P1
(43) Pub. Date: Jan. 5, 2017

(54) Phalaenopsis orchid plant named 'PHALDEMCEP'

(50) ;

(71) Applicant: ANTHURA B.V., Bleiswijk (NL)

(72) Inventor: Martinus Nicolaas Gerardus Van Swieten, Leimuiden (NL)

(21) Appl. No.: 14/545,879

(22) Filed: Jul. 1, 2015

(51) Int. Cl. A01H 5/00

(52) U.S. Cl. PLT/311

(57) ABSTRACT

A new and distinct variety of Phalaenopsis plant named ‘PHALDEMCEP’, particularly characterized by having white flowers, 2 to 5 peduncles, an inflorescence that is long and moderate, leaves that are narrow and obovate, and is propagated by meristem tissue culture is disclosed.

### GENUS AND SPECIES

Phalaenopsis hybrid

### VARIETY DENOMINATION

‘PHALDEMCEP’

### BACKGROUND OF THE NEW PLANT

The present invention comprises a new and distinct cultivar of Phalaenopsis plant, botanically known as Phalaenopsis of the Orchidaceae family, and hereinafter referred to by the cultivar name ‘PHALDEMCEP’.

Phalaenopsis comprises a genus of about 60 species of herbaceous perennials many of which, or the hybrids thereof, are suitable for cultivar in the home or greenhouse. Phalaenopsis is predominantly epiphytic or rock dwelling, and is native to tropical Asia, the Malay Archipelago, and Oceania. The species typically has 2-ranked, fleshy, oblong or elliptic leaves affixed to a short central stem (monopodial growth), which vary in size from 12 to 20 cm to over 60 cm. The leaves may be entirely green or mottled with silver grey.

Phalaenopsis orchids, often referred to as ‘Moth Orchids’ in the horticultural trade, are frequently used to furnish cut flowers for the florist trade or sold as flowering potted-plants for home or interiorscape.

Phalaenopsis produces upright or pendent lateral racemes or panicles, often with many showy flowers which open in succession beginning with the lowermost. The flowers possess three sepals and three petals; the lateral ones being alike. The lowermost petals, called the labellum, are three-lobed and are often more brightly-colored than the other flower segments. Flower colors include various shades of pink, white, yellow, and red-brown.

Phalaenopsis orchids are typically propagated from seeds. Asexual propagation of Phalaenopsis is often done from off-shoots which arise from the lower bracts of the inflorescence. The resulting plants are detached from the mother plants and may be planted in a suitable substrate.

The new Phalaenopsis ‘PHALDEMCEP’ is particularly characterized by its attractive and unique white flowers, economical propagation by tissue culture, rapid growth, and a plant dimension suitable for packaging and shipping to the market.

‘PHALDEMCEP’ is a product of a planned breeding program conducted by the inventor in Bleiswijk, The Netherlands.

The new Phalaenopsis ‘PHALDEMCEP’ originated from a cross made in November 2000 in Bleiswijk, The Netherlands. The female parent is a Phalaenopsis pot plant named ‘07693-0015’ (unpatented), while the male parent is a Phalaenopsis pot plant named ‘07693-0004’ (unpatented). A single plant was selected in February 2007 and has been asexually reproduced repeatedly by meristem tissue culture in Bleiswijk, The Netherlands over a 5-year period. The new variety has been found to retain its distinctive characteristics through successive asexual propagations.

Asexual reproduction of ‘PHALDEMCEP’ by tissue culture was first performed in April 2010 in Bleiswijk, The Netherlands and has demonstrated that the new cultivar is firmly fixed and retained through successive generations of asexual reproduction.

Plant Breeder's Rights for this variety have been applied for in Europe on Apr. 25, 2014. ‘PHALDEMCEP’ has not been made publicly available or sold anywhere in the world more than one year prior to the filing date of this application.

### SUMMARY OF THE INVENTION

The following are the most outstanding and distinguishing characteristics of this new cultivar when grown under normal horticultural practices in Bleiswijk, The Netherlands.

1) White flowers;

2) 2 to 5 peduncles;

3) Inflorescence is long and moderate;

4) Shape of the leaf is narrow and obovate; and

5) Plants are propagated by meristem tissue culture.

### DESCRIPTION OF THE PHOTOGRAPHS

This new Phalaenopsis plant is illustrated by the accompanying photographs which show the overall plant habit including blooms, buds and foliage of the plant; the colors shown are as true as can be reasonably obtained by conventional photographic procedures. The photographs are of a 50-week old plant grown in a greenhouse in Bleiswijk, The Netherlands in April 2015.

FIG. 1 shows the overall plant habit, including blooms, buds and foliage of ‘PHALDEMCEP’.

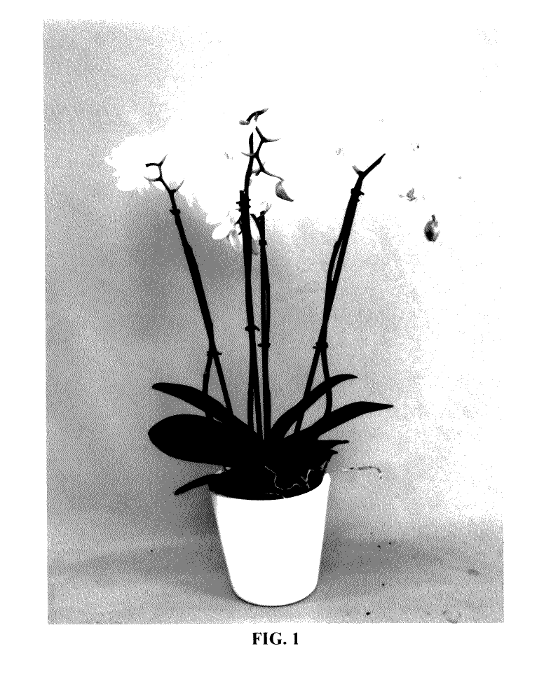

FIG. 2 shows a close-up of a flower of ‘PHALDEMCEP’.

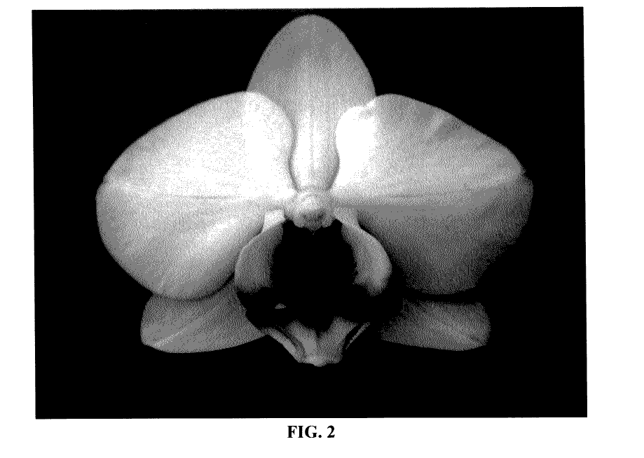

FIG. 3 shows a close-up of the leaves of ‘PHALDEMCEP’.

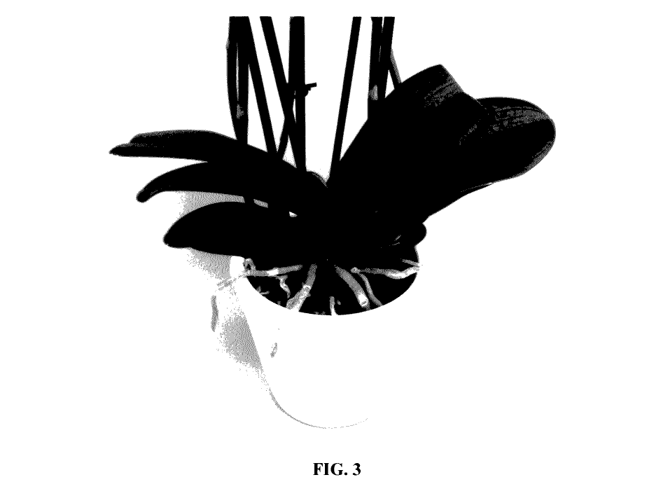

### DESCRIPTION OF THE NEW VARIETY

The following detailed description sets forth the distinctive characteristics of ‘PHALDEMCEP’. The data which define these characteristics were collected from asexual reproductions carried out in Bleiswijk, The Netherlands. The plant history was taken on 50-week old plants which were planted from a nursery tray in 12 centimeter pots and grown in a greenhouse between 27° C. to 29° C. for 30 weeks, continued by a cooling period of 8 weeks between 18° C. to 20° C. and 12 weeks in a greenhouse of 21° C. Observations were made in April 2015. Color readings were taken under 4-6000 lux natural light in the greenhouse. Color references are primarily to The R.H.S. Colour Chart of The Royal Horticultural Society of London (R.H.S.) (2001).

### DETAILED BOTANICAL DESCRIPTION

Classification:
   Family.—Orchidaceae.
   Botanical.—Phalaenopsis hybrid.
   Common name.—Phalaenopsis.
   Variety name.—‘PHALDEMCEP’.
Parentage:
   Female parent.—Phalaenopsis cultivar ‘07693-0015’ (unpatented).
   Male parent.—Phalaenopsis cultivar ‘07693-0004’ (unpatented).
Propagation:
   Type.—Meristem tissue culture.
Plant:
   Crop time (time to produce a finished flowering plant).—48 to 50 weeks for a 12 cm pot.
   Growth habit of peduncle.—Standard, green leaves, raceme to panicle.
   Height (including pot, including inflorescence).—50.0 cm to 60.0 cm.
   Width (measured from leaf tips).—28.0 cm to 33.0 cm.
   Vigor.—Moderate.
Roots:
   Root description.—Grey-green-colored roots with branching lateral roots having green-light purple colored root tips.
Leaves:
   Mature leaves.—Quantity per plant: 8 to 10 leaves are produced before flowering Length (fully expanded): 14.0 cm to 18.0 cm Width: 6.0 cm to 7.0 cm Shape: Obovate (see enclosure 1a) Base shape: Moderately elongated (see enclosure 1b) Apex: Unequal obtuse (see enclosure 1c) Leaf blade angle with the petiole (measured from the horizontal position): Between 40 degrees (see enclosure 1d.A) and 70 degrees (see enclosure 1d.B) Leaf margin: Entire Color: Upper surface: RHS 147A Lower surface: RHS 147B Texture: Smooth Thickness: 2.5 mm to 2 7 mm Venation: Pattern: Parallel Color of the midvein: Upper surface: RHS 147A Lower surface: RHS 147B.
Peduncle:
   Quantity per plant.—2 to 5.
   Number of flowers per peduncle.—6 to 10.
   Length.—41.0 cm to 51.0 cm.
   Diameter.—5.1 mm to 5.5 mm.
   Strength.—Moderate.
   Aspect.—Upright.
   Texture.—Smooth.
   Color.—Green (RHS 146A).
   Internode length.—30.0 mm to 40.0 mm.
   Callosities.—None.
Inflorescence description:
   Appearance.—Upright to slightly pendant, raceme to panicle inflorescence with bilaterally symmetrical flowers that open in succession beginning with the lowermost flower.
   Inflorescence size.—Height (from base to tip): 120.0 mm to 170.0 mm Diameter: 3.3 mm to 3.5 mm.
   Flowering time.—First flowers can be expected 10 to 11 months after planting in a 12 cm pot.
   Flower.—Height: 68.0 mm to 73.0 mm Diameter: 77.0 mm to 82.0 mm Depth of lip: 19.0 mm to 21.0 mm (see enclosure 2).
   Flower longevity.—On the plant: 15 to 22 weeks.
   Fragrance.—Absent.
   Flower bud.—Average size: Medium (see enclosure 3a) Length: 1.6 cm to 2.0 cm (see enclosure 3b) Width: 1.4 cm to 1.7 cm (see enclosure 3c) Shape: Egg shape (see enclosure 3d) Color: Green (RHS 145C and 149C) with slightly greyed-purple over color (RHS 186B).
   Petals.—Arrangement: Open/free Shape: Semi-circular (see enclosure 4a) Apex: Emarginated and slightly asymmetric (see enclosure 4b) Margin: Entire Length (from base to tip): 37.0 mm to 39.0 mm Width: 39.0 mm to 41.0 mm Color (when fully opened): Main color: White (RHS 155C) At the base: White (RHS 155C) Over color: Absent.
   Dorsal sepal.—Shape: Elliptic (see enclosure 4a) Apex: Mucronate (see enclosure 4b) Margin: Entire Length (from base to tip): 39.0 mm to 41.0 mm Width: 24.0 mm to 26.0 mm Color (when fully opened): Main color: White (RHS 155C) At the base: White (RHS 155C) Over color: Absent.
   Lateral sepals.—Shape: Ovate (see enclosure 4a) Apex: Obtuse and slightly asymmetric (see enclosure 4b) Margin: Entire Length (from base to tip): 40.0 mm to 42.0 mm Width: 24.0 mm to 26.0 mm Color (when fully opened): Main color: White (RHS 155C) At the base: White (RHS 155C) Over color: Absent.
   Labellum (lip).—Margin: Entire Pubescence on the lip: Absent Whiskers: Present Length of whiskers: Medium (1.0 cm to 1.2 cm) (see enclosure 50 Color of whiskers: White (RHS 155C) with yellow tips (RHS 10B).
   Lateral lobe.—Shape: Type V (as described in the International Union for the Protection of New Varieties of Plants (UPOV) Test Guidelines for Phalaenopsis); spatulate (see enclosure 6a) Length: 20.0 mm to 22.0 mm (see enclosure 6b) Width: 15.0 mm to 17.0 mm (see enclosure 6c) Color: White (RHS 155C) with upper edge green-yellow (RHS 1A) and red-purple stripes towards the callus (RHS 71A).
   Apical lobe.—Shape: Triangular/trullate (see enclosure 7a) Length: 23.0 mm to 25.0 mm (see enclosure 7b) Width: 24.0 mm to 26 0 mm (see enclosure 7c) Color: White (RHS 155C) and yellow toward the callus (RHS 2A).
   Callus.—Average size: Medium (see enclosure 8d) Height: 7.0 mm to 8.0 mm (see enclosure 8a) Length: 5.0 mm to 6.0 mm (see enclosure 8b) Width: 4.0 mm to 4.5 mm (see enclosure 8c) Color: Yellow (RHS 5B) and greyed-orange spots (RHS 173B).
   Reproductive organs.—
   Arrangement.—The stamens, style and stigmas are fused into a single, short structure called the column, possessing one terminal anther with pollen grains united into pollinia, which are covered by an anther cap. The stigma is located under the column behind the pollinia. The ovary is inferior with three carpels present.
   Column.—Length: 9.0 mm to 10.0 mm (see enclosure 9a) Diameter: 5.5 mm to 6.5 mm (see enclosure 9b) Color: White (RHS 155C).
   Pollinia.—Quantity: 2 Size: 1.1 mm to 1.3 mm Color: Orange (RHS 25A).
   Ovary.—Part of the pedicel with small ribs towards the column Length: 9.0 mm to 11.0 mm (see enclosure 9c) Diameter: 2.3 mm to 2 5 mm (see enclosure 9d).
   Pedicel.—Length: 31.0 mm to 33.0 mm (see enclosure 9e) Diameter: 2.7 mm to 2.9 mm (see enclosure 9f) Color: Green (RHS 145B to 145D).
   Disease, pest, and stress resistance.—No specific resistance or susceptibility observed.
   Temperature tolerance.—Tolerant to a low temperature of 15° C. and a high temperature about 30° C.

### Comparison with parental and commercial varieties:

The parental varieties of ‘PHALDEMCEP’ are no longer available for comparison. ‘PHALDEMCEP’ differs from commercial variety ‘PHALBAPAK’ (unpatented) in that ‘PHALDEMCEP’ has a callus with smaller and lighter colored dots and more narrow petals, whereas ‘PHALBAPAK’ has a callus with larger and darker colored dots and slightly wider petals. Additionally, the back of the dorsal sepal of ‘PHALDEMCEP’ is white, whereas the back of the dorsal sepal of ‘PHALBAPAK’ is light lilac.

I claim:

1. A new and distinct variety of Phalaenopsis plant named ‘PHALDEMCEP’, substantially as described and illustrated herein.
